(19) United States
(12) Patent Application Publication
(10) Pub. No.: US 2015/0113860 A1
(43) Pub. Date: Apr. 30, 2015

(54) FUEL COMPOSITIONS COMPRISING HYDROPHOBIC DERIVATIVES OF GLYCERINE

(71) Applicant: ENI S.p.A., Roma (IT)

(72) Inventors: Alberto De Angelis, Legnano (MI) (IT); Giulio Assanelli, Pavia (IT); Paolo Pollesel, San Donato Milanese (MI) (IT)

(73) Assignee: ENI S.p.A., Roma (IT)

(21) Appl. No.: 14/390,824

(22) PCT Filed: Apr. 3, 2013
(86) PCT No.: PCT/IB2013/052654

§ 371 Date: Oct. 6, 2014

(30) Foreign Application Priority Data
Apr. 6, 2012 (IT) MI2012A 000570

(51) Int. Cl. C10L 1/185; C07D 317/12; C10L 10/02; C10L 10/04; C07D 317/22

(52) U.S. Cl. CPC C10L 1/1855; C10L 10/04; C07D 317/22; C10L 10/02; C07D 317/12; C10L 2200/0469; C10L 2270/023; C10L 2270/026
USPC 44/352; 44/350; 549/453; 549/430

(57) ABSTRACT

The object of the present invention relates to a composition that can be used as fuel comprising: at least one hydrocarbon mixture at least one hydrophobic ketal or acetal of glycerine. Said composition can be advantageously used as fuel for diesel or gasoline engines.

The present invention relates to a composition comprising a hydrocarbon mixture and one or more hydrophobic ketals or acetals of glycerine. The above composition can be advantageously used as fuel for diesel or gasoline engines. The present invention also relates to the use of hydrophobic ketals or acetals of glycerine as fuel component. The present invention also relates to a method for the preparation of said hydrophobic compounds. Some of these ketals or acetals are new and are a further object of the present invention.

It is known that the emissions produced by the combustion of fuels of a fossil origin containing carbon dioxide (CO₂), carbon monoxide (CO), nitrogen oxides (NOₓ), sulfur oxides (SOₓ), uncombusted hydrocarbons (HC), volatile organic compounds and particulate (PM), are the cause of environmental problems such as, for example, the production of ozone, the greenhouse effect (in the case of nitrogen and carbon oxides), acid rain (in the case of sulfur and nitrogen oxides).

In recent years, the increase in the cost of crude oil and a maturing awareness with respect to the environmental problems described above, have increased the necessity for finding alternative, biodegradable and renewable energy sources.

Consequently, the progressive substitution of fuels deriving from fossil energy sources such as, for example, coal, petroleum, natural gas, with fuels deriving from alternative energy sources such as, for example, vegetable oils, animal fats, biomasses, algae, is becoming of increasing interest on a worldwide scale and efforts have therefore been made in the art for obtaining new fuels from renewable energy sources.

An oxygenated compound that can also be obtained from renewable sources, commonly added to fuels, is ethanol, which however has the defect of being miscible with water, hygroscopic, and immiscible with gasoil within a wide temperature range: phase separation can therefore take place and the mixtures obtained are unstable as described, for example, by Lapuerta et al. in the article “Stability of diesel-bioethanol blends for use in diesel engines”, published in “Fuel” (2007), Vol. 86, pages 1351-1357. Another alcohol that can also be obtained from renewable sources and used as component to be added to fuels, is butanol, which has a better miscibility with gasoil than that of ethanol: however it is still not satisfactory. At low temperatures, in fact, butanol-gasoil mixtures are not homogeneous. A further problem linked to the use of these alcohols, is the low cetane number of the alcohol-gasoil mixture which causes a high ignition delay in internal compression diesel engines.

The use of biodiesel and hydrotreated vegetable oils (HVO) as such or mixed with gasoil, is also known, as also gasoil mixtures comprising alcohols of a biological origin. Biodiesel generally comprises a mixture of fatty acid alkyl esters, in particular a mixture of fatty acid methyl esters (FAME) and can be produced starting from raw materials of a natural origin containing triglycerides (generally triesters of glycerine with fatty acids with a long alkyl chain). These raw materials as such, or the triglycerides obtained after separating said raw materials, are subjected to a transesterification process in the presence of an alcohol, in particular methanol, and a catalyst, so as to obtain said fatty acid alkyl esters, in particular said fatty acid methyl esters (FAME).

The use of said methyl esters of fatty acids (FAME) as such or in a mixture with gasoil, however, has various problems relating to the stability to oxidation and, in addition, during the synthesis of FAME, there is the formation of glycerine (about 10% by weight), as by-product, whose use is an important aspect for upgrading the production process of biodiesel.

The use is also known of hydrotreated vegetable oils (HVO), also known as green diesel, which are produced by hydrogenation/deoxygenation of a material deriving from renewable sources such as, for example, soybean oil, rape oil, corn oil, sunflower oil, comprising triglycerides and free fatty acids, in the presence of hydrogen and a catalyst as described, for example, by Holmgren J. et al. in the article “New developments in renewable fuels offer more choices”, published in “Hydrocarbon Processing”, September 2007, pages 67-71. In this article, the best characteristics of said hydrotreated vegetable oils (HVO) are indicated, with respect to fatty acid methyl esters (FAME), in particular, in terms of improved oxidative stability and improved cold properties. Furthermore, said hydrotreated vegetable oils (HVO) do not have the problem of greater emissions of nitrogen oxides (NOₓ).

Due to the lack of oxygen atoms in said hydrotreated vegetable oils (HVO), however, their use in diesel engines mixed with gasoil in an amount lower than 5% by volume with respect to the total volume of said blend, does not provide significant benefits with respect to particulate emissions (PM).

The necessity is therefore felt of finding new compositions that can be used as fuel, in which there is a component deriving from renewable energy sources.

The necessity is also felt of exploiting glycerine, whose market is currently saturated, as starting material for obtaining compounds of a biological origin which can provide high performances as hydrocarbon components.

At present, one of the possible uses of glycerine is to make it react through an etherification reaction with olefins to give the corresponding ethers, useful as oxygenated components for gasoline and diesel. The most widely-used olefin, object of numerous patents is isobutene. Reaction with isobutene leads to the formation of tert-butyl ethers of glycerine, of which the most interesting is di-tert-butyl-ether.

US 2007/0283619 describes a process for the production of biofuels by the transformation of triglycerides into at least two groups of biofuels containing monoesters of fatty acids and soluble ethers or acetals of glycerine. Said ethers and acetals of the known art, however, have a high affinity to water and a low miscibility with the hydrocarbon phase: this is a serious limitation for use as fuel component, as significant quantities of water can be dissolved in the fuel mixture containing said acetals, with serious damage to the engine of the motor vehicle due to corrosion phenomena. Furthermore, the presence of substances miscible with water in gasolines leads to the formation of formaldehyde, a carcinogenic substance, in the emissions (B. Strus et al., Fuel 87 (2008), 957-963, ELSEVIER).

The Applicant has now found that the addition of particular hydrophobic acetals and/or ketals deriving from glycerine, allow a composition to be obtained which can be advantageously used as fuel, in particular as fuel for both diesel and gasoline engines.

An object of the present invention therefore relates to compositions that can be used as fuels, or fuel components, containing:
a hydrocarbon mixture
at least one cyclic acetal or ketal selected from those having formula (I) and (II):

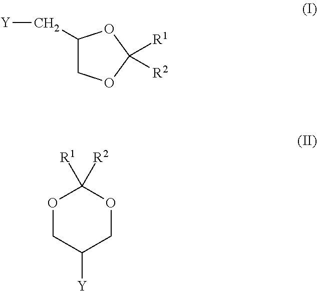

wherein
R¹ is a linear or branched alkyl containing from 1 to 6 carbon atoms, possibly substituted by an alkoxide group OR, wherein R is an alkyl containing from 1 to 4 carbon atoms,
R² is H or a linear or branched alkyl containing from 1 to 6 carbon atoms, possibly substituted by an alkoxide group OR, R being an alkyl containing from 1 to 4 carbon atoms,
R² is the same or different from R¹,
Y is selected from H or OR³, R³ being a linear or branched alkyl containing from 1 to 8 carbon atoms.
R² is preferably selected from H, CH₃; C₂H₅ and R¹ is selected from CH₃, C₂H₅, C₃H₇, C₄H₉. R³ preferably contains from 2 to 4 carbon atoms. Even more preferably R³ can be selected from ethyl, n-butyl, isobutyl, 3-methyl-1-butyl and 2 methyl-1-butyl and, even more preferably is ethyl or n-butyl.

Said compounds (I) and (II) are prepared starting from glycerine and provide high performances as fuel components, overcoming the known problems of acetals, relating to their high affinity with water and low affinity with the remaining hydrocarbon component of the fuel. Compounds having formula (I) and (II), alone or mixed with each other, can therefore be advantageously used as fuel components, in particular gasoil, especially for automotive use, and as additives for gasoline, and their addition to gasoil or gasoline allows, inter alia, a significant reduction in particulate emissions. The composition containing them is less sensitive to water and consequently corrosion phenomena in engines using this type of compositions are considerably reduced. The addition of compounds having formula (I) and (II), moreover, has no negative influence on the characteristics of the starting gasoil, such as, for example, the cold properties, cloud point (CP) and cold filter plugging point (CFPP), nor does it negatively influence either the demulsibility and lubricity characteristics of the composition or oxidation stability of the starting gasoil.

According to a preferred embodiment of the present invention, said compounds having formula (I) and (II) can be present in said composition in quantities ranging from 0.5% by volume to 15% by volume, preferably from 1% by volume to 10% by volume with respect to the total volume of said composition, wherein said quantities, when in the presence of two acetals and/or ketals, refer to the sum of their volumes.

For the purposes of the present invention, any hydrocarbon mixture can be used as fuel. In particular, the hydrocarbon mixture can be selected from gasoil, gasoline, biodiesel, green diesel and mixtures thereof.

In particular, said gasoil can be selected either from gasoils which fall within gasoil specifications for motor vehicles according to the standard EN 590:2009, or gasoils which do not fall within these specifications. Gasoil is generally a mixture containing hydrocarbons such as, for example, paraffins, aromatic hydrocarbons and naphthenes, typically having from 9 to 30 carbon atoms. The distillation temperature of gasoil generally ranges from 160 to 450° C. According to a preferred embodiment of the present invention, said gasoil can have a density at 15° C., determined according to the standard EN ISO 12185:1996/01:2001, ranging from 780 kg/m³ to 845 kg/m³, preferably from 800 kg/m³ to 840 kg/m³. According to a further preferred embodiment of the present invention, said gasoil can have a flash point, determined according to the standard EN ISO 2719:2002 higher than or equal to 55° C., preferably higher than or equal to 65° C. According to a preferred embodiment of the present invention, said gasoil can have a cetane number, determined according to the standard EN ISO 5165:1998, or the standard ASTM D6890:2008, higher than or equal to 47, preferably higher than or equal to 51.

Gasoils that can be conveniently used in the compositions of the present invention can therefore be all known gasoils and can also derive from the mixing of diesel cuts of different origins and having different compositions. The sulfur content of these diesel cuts preferably ranges from 2,000 to 50 mg/kg, and even more preferably from 50 to 3 mg/kg.

Typical diesel cuts can be medium distillates, preferably having a boiling point ranging from 180 to 380° C. Examples of these cuts can be gasoils from primary distillation, gasoils from vacuum distillation, and thermal or catalytic cracking, such as for example, desulfurized gasoil cuts coming from fluid bed catalytic cracking (light cycle oil (LCO)), fuels from a Fischer-Tropsch process or of a synthetic origin. Cuts obtained from these after hydrogenation treatment can also be conveniently used.

If the hydrocarbon mixture is a gasoline, gasolines characterized by a T95 (ASTM D86) not higher than 250° C., preferably not higher than 240° C., can be conveniently used, wherein T95 refers to the temperature at which 95% by volume of gasoline distills. Gasolines with T95 lower than 250° C., in particular lower than 240° C., having a density ranging from 855 to 910 kg/m³, are preferably used. Gasolines that can be conveniently used are those deriving from catalytic processes, preferably deriving from fluid bed catalytic cracking (FCC) processes, reforming processes, and mixtures thereof. In particular, HCN gasolines are therefore used, i.e. heavy gasolines (initial boiling point 150° C.) from FCC as such or desulfurized, and gasolines called Heavy reformates, i.e. heavy gasolines (initial boiling point 150° C.) from reforming, or mixtures thereof.

The sulfur content of these gasoline cuts ranges from 2000 to 50 mg/kg, and even more preferably from 50 to 1 mg/kg.

If the composition contains a biodiesel, said biodiesel, as previously mentioned, comprises a mixture of fatty acid alkyl esters, in particular a mixture of fatty acid methyl esters (FAME) and can be produced starting from raw materials of a natural origin containing triglycerides (generally triesters of glycerine with fatty acids having a long alkyl chain) such as, for example, crude vegetable oils obtained by pressing the seeds of oleaginous plants such as, for example, rape, palm, soybean, sunflower, mustard, in addition to other sources of triglycerides such as, for example, algae, animal fats, or used or waste vegetable oils. These raw materials as such, or the triglycerides obtained after subjecting said raw materials to separation, are subjected to a transesterification process in the presence of an alcohol, in particular methanol, and a catalyst, so as to obtain said fatty acid alkyl esters, in particular said fatty acid methyl esters (FAME). Further details relating to the production of biodiesel are provided for example in Hanna et al., in the review “Biodiesel production: a review”, published in “Bioresource Technology” (1999), vol. 70, pages 1-15. Said biodiesel is preferably selected from those that fall within the specifications of biodiesel for motor vehicles according to the standards EN 14214:2008.

The composition can contain hydrotreated vegetable oils, called “green diesel”: they are produced by the hydrogenation/Deoxygenation of a material deriving from renewable sources such as, for example, soybean oil, rape oil, corn oil, sunflower oil, comprising triglycerides and free fatty acids, in the presence of hydrogen and a catalyst as described for example by Holmgren J. et al. in the article “New developments in renewable fuels offer more choices”, published in “Hydrocarbon Processing”, September 2007, pages 67-71.

The compositions of the present invention are prepared by mixing the single components. Possible other additives present in the final composition can be introduced into both the final composition itself or into the hydrocarbon mixture before mixing.

The compounds having formula (I) and (II) are prepared starting from glycerine by means of a process comprising the following steps:
1) transformation of glycerine into propanediol or alkoxy-propanediol
2) reaction of the diol obtained in step (1) with a carbonyl compound selected from aldehydes and ketones to give the corresponding cyclic acetal having formula (I) or (II).

In particular, the transformation of step (1) can therefore correspond to the etherification of glycerine, wherein said etherification is effected by reaction with an alcohol having formula R³OH to give the corresponding 3-alkoxy-1,2-propanediol, 2-alkoxy-1,3-propanediol or a mixture thereof, wherein alkoxy=R³O.

The etherification step (1) can be effected according to any of the known methods for the preparation of ethers. It is possible, for example, to react glycerine in the presence of the alcohol and an acid catalyst. Acid catalysts that can be conveniently used are, for example, acid exchange resins, acid zeolites, silico-aluminas, supported phosphoric acid. Solvents that can be conveniently used are preferably the same alcohols with which the corresponding ether is to be formed. The reaction is preferably carried out at a temperature ranging from 50 to 200° C., and at a pressure ranging from 1 to 20 atmospheres. The space velocity preferably ranges from 0.1 to 20 hours⁻¹. The alcohol/glycerine molar ratio preferably ranges from 1 to 10. Etherification in position 1 is favoured, using a fixed bed reactor, by the choice of low contact times. Etherification in position 2, on the other hand, is favoured by the choice of high contact times and/or high R³OH/glycerine molar ratios. Intermediate conditions allow a mixture containing both 3-(R³O)-1,2-propanediole and 2-(R³O)-1,3-propanediol to be obtained.

R³OH alcohols that can be conveniently used for preparing the alkoxides of step (1) are alcohols in which R³ is a linear or branched alkyl containing from 1 to 8 carbon atoms, preferably from 2 to 4 carbon atoms. Alcohols that can be conveniently used are therefore ethanol, n-butanol, iso-butanol, 3-methyl-1-butanol, 2-methyl-1-butanol, or mixtures thereof. Ethanol or n-butanol are preferred.

Alcohols are preferably used, that can also be obtained biologically, i.e. for example by the fermentation of biomasses or derivatives of biomasses, or by the fermentation of biomasses deriving from agricultural crops rich in carbohydrates and sugars, or by the fermentation of lignocellulosic biomasses, or by the fermentation of algal biomasses. The lignocellulosic biomass can derive from agricultural crops rich in carbohydrates and sugars such as, for example, corn, sorghum, barley, beet, sugar cane, or mixtures thereof. The lignocellulosic biomass can be selected, for example, from:
products of crops specifically cultivated for energy use (such as, for example, miscanthus, foxtail millet, switchgrass, common cane), including waste products, residues and scraps of said crops or their processing;
products of agricultural cultivations, forestation and silviculture, comprising wood, plants, residues and waste products of agricultural processings, forestation and silviculture;
waste of agro-food products destined for human nutrition or zootechnics;
residues, not chemically treated, of the paper industry;
waste products coming from the differentiated collection of solid urban waste (such as, for example, urban waste of a vegetable origin, paper, etc.);

or mixtures thereof.

The alcohol used can, for example, also derive from the fermentation of at least one algal biomass cultivated for energy purposes, or the fermentation of residues or derivatives from the cultivation of said biomass.

The fermentation can be carried out in accordance with the methods of the known art. Said fermentation, for example, can be carried out in the presence of natural microorganisms, or genetically modified microorganisms in order to improve said fermentation.

The transformation of step (1) can correspond to a reduction in the glycerine, in this case the corresponding diol can be obtained: said reduction is carried out with hydrogen in the presence of a suitable catalyst and leads to the formation of 1,2-propanediol, 1,3-propanediol, or mixtures thereof.

The reduction step (1) can be carried out according to any of the known methods for the reduction of hydroxyl groups. It is possible, for example, to react glycerine with hydrogen in the presence of a reduction catalyst. Reduction catalysts that can be used can all be known reduction catalysts. Copper chromite, mixed chromium-zinc-copper oxides, noble metals on coal, noble metals on iron oxide, can be used for example, and more preferably palladium on coal, platinum on coal and palladium on iron oxide, the latter catalyst being preferred.

Solvents that can be used are linear aliphatic alcohols or the same diol to be obtained as product. The reduction reaction can be carried out at a temperature ranging from 100° C. to 250° C., under a hydrogen pressure ranging from 1 to 100 atmospheres.

Mixtures of 1,2-propanediol and 1,3-propanediol, are obtained, in which 1,2-propanediol is always predominant. The mixture can be used as such for the subsequent step or subjected to separation, for example by distillation, in order to isolate the desired diol.

Greater details relating to reduction processes that can be used in the preparation process of the present invention are described, for example, in the article Selective hydrogenolysis of glycerol promoted by palladium, Green Chemistry 2009, 111, 1511-13.

In step (2), the product resulting from step (1), whether it be propanediol or alkoxy-propanediol, is reacted with an aldehyde or ketone having formula R¹COR², wherein:
R¹ is a linear or branched alkyl containing from 1 to 6 carbon atoms, possibly substituted by an alkoxide group OR, wherein R is an alkyl containing from 1 to 4 carbon atoms,
R² is H or a linear or branched alkyl containing from 1 to 6 carbon atoms, possibly substituted by an alkoxide group OR, R being an alkyl containing from 1 to 4 carbon atoms,
R² is the same as or different from R¹.

The corresponding ketals or acetals having formula (I) or (II) are obtained, wherein R¹ and R² have the same meanings indicated above and Y is H or OR³, R³ being a linear or branched alkyl containing from 1 to 8 carbon atoms.

In particular, a cyclic acetal with 5 (1,3-dioxolane) substituted by groups R¹, R² and, respectively, by an alkoxymethyl group or a methyl group (formula I), is obtained from 3-alkoxy-1,2-propanediol and 1,2-propanediol.

A cyclic ketal or acetal with 6 atoms (1,3-dioxane) substituted by groups R¹, R² and with an alkoxyl group or hydrogen (formula II) respectively, in position 5, is obtained from 2-alkoxy-1,3-propanediol and 1,3-propanediol.

R¹ is preferably selected from H, CH₃; C₂H₅ and R² is selected from CH₃, C₂H₅, C₃H₇, C₄H₉.

In particular, it is preferable for the aldehyde or ketone to be selected from acetaldehyde, acetone, propionaldehyde, butanal, 1-ethoxyacetone, 1-butoxyacetone.

According to a preferred aspect, the aldehydes and the ketones of a biological origin are used, such as, for example:
acetaldehyde, obtainable by oxidation from ethanol of a biological origin, according to the known methods;
propanal obtainable according to the known methods by dehydration from 1,2-propanediol, in turn obtained by hydrogenation of glycerine of a biological origin;
ethoxyacetone, obtainable for example by etherification, with ethanol of a biological origin, of hydroxyacetone, in turn obtained by treatment of glycerine of a vegetable origin on copper chromite, according to what is described in Dehydration of glycerol to acetol via catalytic reactive distillation, C. Chiu, M. A. Dasari, G. J. Suppes, W. R. Sutterlin, AIChE Journal, October 2006, 52 (10), 3543-48;
acetone, obtainable from fermentative processes of sugars or by reaction of ethanol of a biological origin on base catalysts, according to the known methods;
butanal obtained by oxidation, according to the known methods, of butanol of a biological origin.

The reaction for the formation of the acetal or ketal is carried out in the presence of an acid catalyst, according to the known formation methods of acetals or ketals.

Catalysts that can be conveniently used are acid exchange resins, zeolites, silico-aluminas. Solvents that can be used are the same ketones or aldehydes, used in excess. The molar ratio between aldehyde, or ketone, and diol preferably ranges from 1/1 to 10/1 and even more preferably from 3/1 to 5/1. Reaction temperatures that can be conveniently used range from −10° C. to 120° C. and even more preferably from 20 to 80° C.

Greater details relating to formation processes of acetals that can be used in the preparation process of the present invention are described, for example, in J. Deutsch, A. Martin, H. Lieske, Investigation on heterogenuously catalysed condensation of glycerol to cyclic acetals, Journal of catalysis, 245, 2007, 428-35.

The following acetals are new and are a further object of the present invention:

2,4-diethoxymethyl-2-methyl-1,3-dioxolane, 2-ethoxymethyl-2,4-dimethyl-1,3-dioxolane, 2-ethyl-4-ethoxymethyl-1,3-dioxolane.

The fuel composition, object of the present invention, can optionally comprise conventional additives known in the art such as, for example, flow improvers, lubricity improvers, cetane improvers, antifoam agents, detergents, antioxidants, anticorrosion agents, antistatic additives, dyes, or mixtures thereof. Said additives, if present, are generally present in a quantity not higher than 0.3% by volume with respect to the total volume of said composition which is equal to 100.

Some illustrative and non-limiting examples are provided hereunder for a better understanding of the present invention and for its practical embodiment.

### EXAMPLE 1

### Synthesis of the Acetal Having Formula (I) Wherein R¹═R²═CH₃ and Y═OCH₂CH₃ (2,2-dimethyl-4-ethoxymethyl-1,3-dioxolane)

A catalyst consisting of a commercial acid resin (Amberlyst 36) is charged into a fixed-bed reactor heated to a temperature of 90° C. and a mixture of glycerine and ethanol are fed in a molar ratio 1/6, at a temperature of 180° C. and a space velocity of 0.5 hours⁻¹. A conversion of glycerine equal to 70% is obtained, with a total selectivity to 3-ethoxy-1,2-propanediol, monoethoxyether of glycerine (MEP). The monoethoxypropanediol thus obtained is separated from the non-reacted glycerine by distillation. 10 g of the previous MEP are introduced into a glass flask and acetone is added, in a molar ratio MEP/acetone=1/6. 5 g of commercial acid resin (Amberlyst 36) are added and the reaction mixture is heated to a temperature of 50° C., under stirring for two hours. At the end of this period, the resulting product is analyzed, obtaining a conversion of MEP equal to 67% and a selectivity to the desired product 2,2-dimethyl-4-ethoxymethyl-1,3-dioxolane, equal to 100%. The product obtained is separated from the non-reacted components by distillation which allows it to be obtained as pure product.

### EXAMPLE 2

### Test

The acetal obtained in Example 1 is mixed with an equal volume of distilled water and the mixture is stirred for 6 hours to reach equilibrium. The acetal of Example 1 proves to be immiscible in water from which it can be easily separated by means of a separating funnel. After separation, the water content in the organic phase consisting of the acetal of Example 1, is determined by means of Karl-Fisher titration. The water content proves to be equal to 1.7% by weight. This ketal can therefore be used as fuel component of a biological origin as it has a negligible affinity with respect to water.

### EXAMPLE 3

### Comparative

### Synthesis of 2,2 dimethyl-4-hydroxymethyl-1,3-dioxolane

10 g of glycerine are introduced into a glass flask and acetone is added in a molar ratio glycerine/acetone=1/6. 5 g of commercial acid resin (Amberlyst 36) are added and the reaction mixture is heated to a temperature of 80° C., under stirring for two hours. At the end of this period, the resulting product is analyzed: the conversion of glycerine is equal to 70% and the selectivity to the desired product 2,2-dimethyl-4-hydroxymethyl-1,3-dioxolane, is equal to 100%. The product obtained is separated from the non-reacted components by distillation which allows it to be obtained as pure product.

### EXAMPLE 4

### Comparative Test

The same procedure is effected as in Example 2, using the acetal obtained in Example 3. The acetal of Example 3 is completely mixed with the water, forming a single phase. This ketal cannot therefore be used as fuel component as it shows an extremely high affinity with respect to water.

### EXAMPLE 5

### Synthesis of the Acetal Having Formula (I) Wherein R¹═R²═CH₃ and Y═H (2,2,4-trimethyl-1,3-dioxolane)

10 g of 1,2 propanediol are introduced into a glass flask and acetone is added in a molar ratio propanediol/acetone=1/6. 5 g of commercial acid resin (Amberlyst 36) are added and the reaction mixture is heated to a temperature of 50° C., under stirring for two hours. At the end of this period, the resulting product is analyzed: the conversion of propanediol is equal to 75% and the selectivity to the desired product 2,2,4-trimethyl-1,3-dioxolane, is equal to 100%. The product obtained is separated from the non-reacted components by distillation.

### EXAMPLE 6

### Test

The acetal obtained in Example 5 is mixed with an equal volume of distilled water and the mixture is stirred for 6 hours to reach equilibrium: the acetal proves to be immiscible in water from which it can be easily separated by means of a separating funnel. After separation, the water content in the organic phase consisting of the acetal of Example 5, is determined: the water content measured by means of Karl-Fisher titration, proves to be equal to 2.6%. This ketal can therefore be used as fuel component of a biological origin as it has a very low affinity with respect to water.

1. Compositions containing:
a hydrocarbon mixture
at least one cyclic ketal or acetal selected from those having formula (I) and (II): 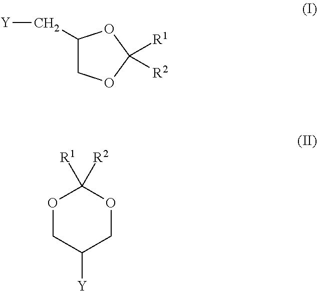
wherein
R¹ is a linear or branched alkyl containing from 1 to 6 carbon atoms, possibly substituted by an alkoxide group OR, wherein R is an alkyl containing from 1 to 4 carbon atoms,
R² is H or a linear or branched alkyl containing from 1 to 6 carbon atoms, possibly substituted by an alkoxide group OR, wherein R is an alkyl containing from 1 to 4 carbon,
R² is the same as or different from R¹,
Y is selected from H or OR³, R³ being a linear or branched alkyl containing from 1 to 8 carbon atoms.

2. The composition according to claim 1, wherein the cyclic ketal and/or acetal having formula (I) or (II) is present in said composition in an amount ranging from 0.5% by volume to 15% by volume.

3. The composition according to claim 2, wherein the cyclic ketal and/or acetal having formula (I) or (II) is present in an amount ranging from 1% by volume to 10% by volume, with respect to the total volume of the composition.

4. The composition according to claim 1, wherein the hydrocarbon mixture is selected from gasoil, gasoline, biodiesel, green diesel and mixtures thereof.

5. The composition according to claim 1, wherein, in the compounds having formula (I) or (II), R² is selected from H, CH₃ and C₂H₅, and R¹ is selected from CH₃, C₂H₅, C₃H₇ and C₄H₉.

6. The composition according to claim 1, wherein, in the compounds having formula (I) or (II), R³ contains from 2 to 4 carbon atoms.

7. The composition according to claim 1, wherein, in the compounds having formula (I) or (II), R³ is ethyl or n-butyl.

8. A process for preparing cyclic ketals and/or acetals having formula (I) or (II) starting from glycerine comprising the following steps:
(1) transformation of glycerine into propanediol or alkoxy-propanediol;
(2) reaction of the diol obtained in step (1) with a carbonyl compound selected from aldehydes and ketones to give the corresponding cyclic acetal having formula (I) or (II): 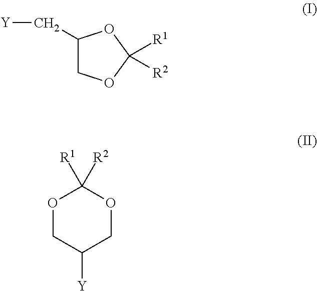
wherein
R¹ is a linear or branched alkyl containing from 1 to 6 carbon atoms, possibly substituted by an alkoxide group OR, wherein R is an alkyl containing from 1 to 4 carbon atoms,
R² is H or a linear or branched alkyl containing from 1 to 6 carbon atoms, possibly substituted by an alkoxide group OR, wherein R is an alkyl containing from 1 to 4 carbon,
R² is the same as or different from R¹,
Y is selected from H or OR³, R³ being a linear or branched alkyl containing from 1 to 8 carbon atoms.

9. The process according to claim 8, wherein the transformation in step (1) corresponds to the etherification of glycerine, effected by reaction with an alcohol having formula R³OH to give the corresponding 3-(R³O)-1,2-propanediol, 2-(R³O)-1,3-propanediol or a mixture thereof, wherein R³ is a linear or branched alkyl containing from 1 to 8 carbon atoms.

10. The process according to claim 8, wherein the transformation in step (1) corresponds to the reduction of glycerine to the corresponding diol, effected with hydrogen in the presence of a catalyst to give 1,2-propanediol, 1,3-propanediol, or mixtures thereof.

11. The process according to claim 8, wherein in step (2), the product resulting from step (1) is reacted with an aldehyde or a ketone having formula R¹COR², wherein:
R¹ is a linear or branched alkyl containing from 1 to 6 carbon atoms, possibly substituted by an alkoxide group OR, wherein R is an alkyl containing from 1 to 4 carbon atoms,
R² is H or a linear or branched alkyl containing from 1 to 6 carbon atoms, possibly substituted by an alkoxide group OR, wherein R is an alkyl containing from 1 to 4 carbon atoms, and
R² is the same as or different from R¹.

12. Cyclic acetals and ketals having formula 2,4-diethoxymethyl-2-methyl-1,3-dioxolane, 2-ethoxymethyl-2,4-dimethyl-1,3-dioxolane, 2-ethyl-4-ethoxymethyl-1,3-dioxolane.

13. Use of the composition according to claim 1 as fuel or fuel component.

14. Use, as fuel components, of one or more cyclic acetals or ketals having formula (I) and (II): 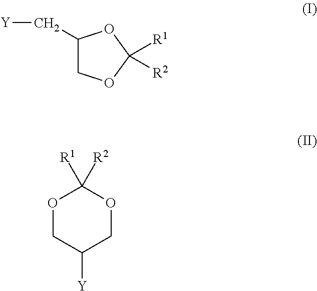
wherein
R¹ is a linear or branched alkyl containing from 1 to 6 carbon atoms, possibly substituted by an alkoxide group OR, wherein R is an alkyl containing from 1 to 4 carbon atoms,
R² is H or a linear or branched alkyl containing from 1 to 6 carbon atoms, possibly substituted by an alkoxide group OR, wherein R is an alkyl containing from 1 to 4 carbon atoms,
R² is the same as or different from R¹,
Y is selected from H or OR³, R³ being a linear or branched alkyl containing from 1 to 8 carbon atoms.
